(19) United States
(12) Patent Application Publication
(10) Pub. No.: US 2024/0050501 A1
(43) Pub. Date: Feb. 15, 2024

(54) Method for Forming Fungi Fermentation Product Containing N-Acetylglucosamine to Convert Cancer Cells into Normal Cells Through Mesenchymal Epithelial Transition Mechanism

(71) Applicant: Kun-Lin Yang, Tainan City (TW)

(72) Inventors: Kun-Lin Yang, Tainan City (TW); Tzu-Yang Yang, Tainan City (TW); Tzu-Chi Yang, Tainan City (TW); Tzu-Yi Yang, Tainan City (TW)

(21) Appl. No.: 18/187,797

(22) Filed: Mar. 22, 2023

(30) Foreign Application Priority Data
Aug. 10, 2022 (CN) 2022109553014

(51) Int. Cl. A61K 36/07; A23L 31/00; C12N 1/14

(52) U.S. Cl. CPC A61K 36/07; A23L 31/00; C12N 1/14

(57) ABSTRACT

The invention discloses a method for forming a fungi fermentation product containing N-acetylglucosamine. The fungi fermentation product containing N-acetylglucosamine can convert cancer cells into normal cells through a mesenchymal epithelial transition mechanism. The method uses fungi as a starting material which together with a Cordyceps-related fungus is fermented to form a fermentation product which induces a mesenchymal epithelial transition to convert cancer cells into normal cells. The fungi cell wall contains chitin. The fermentation liquid is produced by fermentation of Cordyceps with chitin as the substrate. The main product is N-acetylglucosamine confirmed by an HPLC analysis. According to the experimental data, the fermentation product of N-acetylglucosamine converts cancer cells into epithelial cells that contain E-cadherin and has a square shape. Thus, it is proved that the fermented substance converts cancer cells into normal cells through induction of the mesenchymal epithelial transition mechanism.

### FIELD OF THE INVENTION

The invention relates to a method for forming a fungi fermentation product containing N-acetylglucosamine to convert cancer cells into normal cells through a mesenchymal epithelial transition mechanism, which is a new use of biological material.

### DESCRIPTION OF THE RELATED ART

Fungi crops, such as shiitake mushrooms, enoki mushrooms, and black fungus, are currently used for human consumption. However, the value of the fungi crops is not spread, thereby limiting the biological application potential of the fungi crops.

In view of the above-mentioned disadvantage, the inventor provides a method for forming a fungi fermentation product containing N-acetylglucosamine to convert cancer cells into normal cells through a mesenchymal epithelial transition mechanism so as to enhance the efficacy of the product.

### BRIEF SUMMARY OF THE INVENTION

The main purpose of the present invention is to provide a method for forming a fungi fermentation product containing N-acetylglucosamine to convert cancer cells into normal cells through a mesenchymal epithelial transition mechanism, thereby providing a use that can greatly increase the added value of the fungi.

In order to achieve the above purpose, the method for forming a fungi fermentation product containing N-acetylglucosamine to convert cancer cells into normal cells through a mesenchymal epithelial transition mechanism comprises the following steps:

a fungus treatment procedure including preparing a fruiting body or mycelium of fungi crops with a predetermined weight and cleaning the fruiting body, then mixing the fungi crops with water with a predetermined proportion, then crushing the fungi 10 crops to form a fungi liquid, then heating and sterilizing the fungi liquid to form a fungi fruiting body or mycelium liquid, wherein the fungi crops include Lentinula edodes, Agaricus bisporus, Agaricus campestris, Ganoderma lucidum, Flammulina velutipes or Auricularia auricula-judae;

a strain activation procedure including preparing a Cordyceps fungus such as Cordyceps militaris or Ophiocordyceps sinensis, then in turn culturing the Cordyceps fungus under a solid state and a liquid state successively to form a Cordyceps fungus liquid;

a fermentation procedure including mixing and culturing the fungi fruiting body or mycelium liquid and the Cordyceps fungus liquid under a proper condition to form the fungi fermentation product containing N-acetylglucosamine.

The human liver cancer cells (or Huh7) are mesenchymal cells that are transformed from epithelial cells through epithelial-mesenchymal transitions (EMT). That is, the liver cancer cells are mesenchymal cells that are transformed originally from epithelial cells. The epithelial cells have a square or polygonal shape and contain E-cadherin so that the cells are tightly adhered and are not easy to move. The Huh7 cells are transformed into the mesenchymal cells that have an elongated shape and do not contain E-cadherin so that the cells are easy to move.

When the Huh7 cells are cultivated by the fungi fermentation product of the present invention, the profile of the Huh7 cells is transformed from an elongated shape into a square or polygonal shape, and the E-cadherin is restored. Thus, it is proved that, the fungi fermentation product of the present invention induces mesenchymal-epithelial transitions (MET), so that Huh7 cells are converted from the mesenchymal cells into the epithelial cells.

Therefore, according to the present invention, the improvement and therapeutic potential of fungi fermented products for related cancers can be further studied, and the added value of fungi can indeed be improved. It is seen that the present invention is a progressive creation.

### BRIEF DESCRIPTION OF THE DRAWINGS

FIG. 1 is a flowchart of a method for forming a fungi fermentation product containing N-acetylglucosamine to convert cancer cells into normal cells through a mesenchymal epithelial transition mechanism according to the present invention.

FIG. 2 is the HPLC analysis result of a standard substance of N-acetylglucosamine.

FIG. 3 is the HPLC analysis result of the fungi fermentation product of the present invention when it was fermented for two weeks.

FIG. 4 is the HPLC analysis result of the fungi fermentation product of the present invention when it was fermented for four weeks.

FIG. 5 is a photomicrograph of the Huh7 cells that are cultured without the fungi fermentation product.

FIG. 6 is a photomicrograph of the Huh7 cells that are cultured by 5% of the fungi fermentation product.

FIG. 7 is a photomicrograph of the Huh7 cells that are cultured by 10% of the fungi fermentation product.

FIG. 8 is a photomicrograph of the Huh7 cells that are cultured by 15% of the fungi fermentation product.

FIG. 9 is a fluorescent photomicrograph of the Huh7 cells that are cultured by 15% of the fungi fermentation product wherein the Huh7 cells are colored.

FIG. 10 is a comparison photo of the Huh7 cells that are cultured for 8 hours, then added with 15% of the fungi fermentation product, and then cultured for 24 hours.

### DETAILED DESCRIPTION OF THE INVENTION

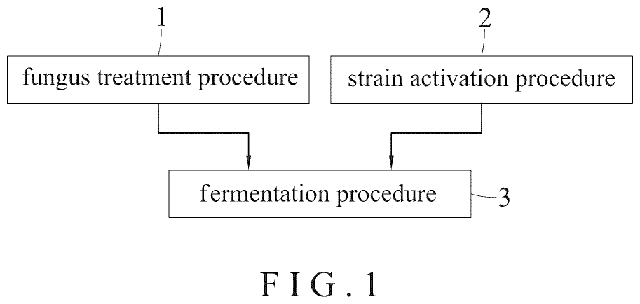

Referring to FIG. 1, the present invention provides a method for forming a fungi fermentation product containing N-acetylglucosamine to convert cancer cells into normal cells through a mesenchymal epithelial transition mechanism.

The method for forming the fungi fermentation product comprises:

a fungus treatment procedure (1) including preparing and cleaning Lentinula edodes stems with a predetermined weight, then mixing the Lentinula edodes stems with water at a weight ratio of 1:9, then crushing the Lentinula edodes stems to form a Lentinula edodes stems liquid, then heating and sterilizing 10% of the Lentinula edodes stems liquid at a temperature of 121° C. during 1 hour;

a strain activation procedure (2) including preparing and placing a Cordyceps militaris (BCRC 33740) in a PDA culture medium to perform a solid-state culture at 25° C. for 7 days, then placing the Cordyceps militaris in a liquid culture medium to perform a liquid-state culture at 25° C. for 10 days, under vibration with a rotation speed of 180 rpm, to form a Cordyceps militaris fungus liquid, and the liquid culture medium has a composition including 20 g of glucose, 1 g of peptone, 20 g of agar and 20 g of malt extract per 1 L of distilled water;

a fermentation procedure (3) including mixing and culturing the Lentinula edodes stems liquid and the Cordyceps militaris fungus liquid at a weight ratio of 20:1 under a temperature of 25° C. for 30 days to form the Lentinula edodes stems fermentation product containing N-acetylglucosamine.

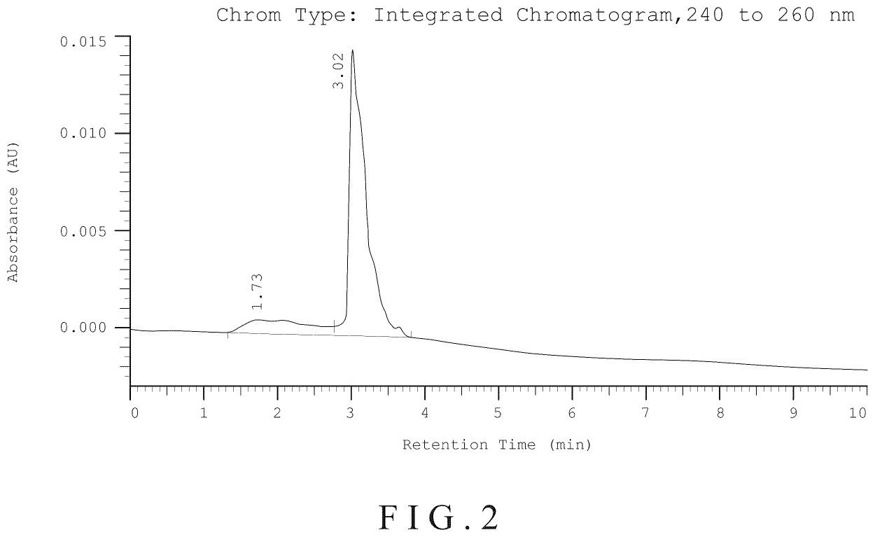

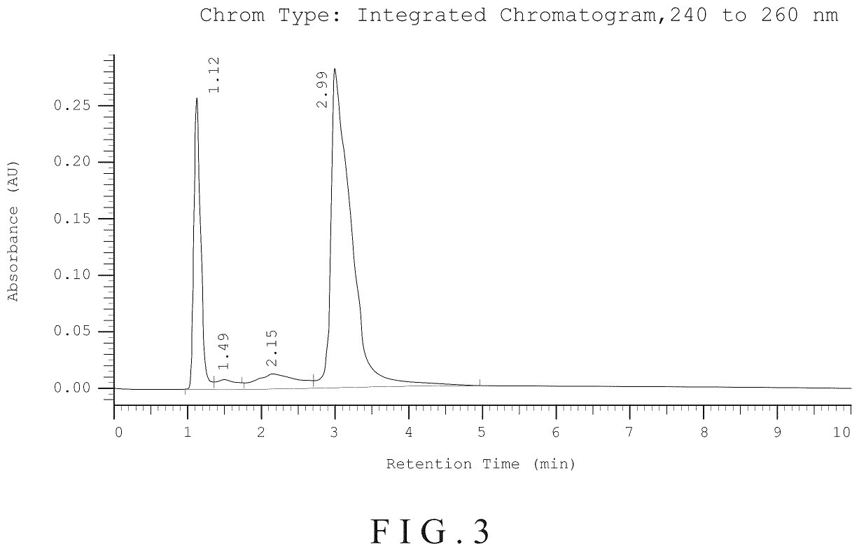

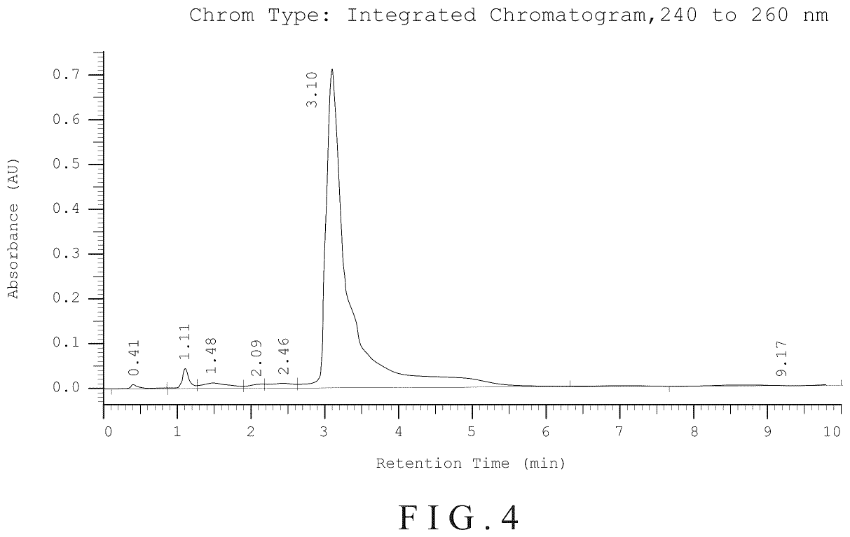

Referring to FIGS. 2-4, in order to understand the composition of the Lentinula edodes stems fermentation product, HPLC (high performance liquid chromatography) is performed on the Lentinula edodes stems fermentation product and the NAG standard product (N-acetylglucosamine) under the same conditions to compare the results. FIG. 2 is the HPLC result of the NAG standard product, and FIGS. 3 and 4 are the HPLC result of the samples of the 14th and 28th day of fermentation of the Lentinula edodes stems fermentation product. It is found that the retention time of the peak of the Lentinula edodes stems fermentation product is the same as that of the NAG standard product. Another peak in the sample on the 14th day shows that there is still a substance with a larger molecular weight at the position of 1 minute of the retention time, but the sample on the 28th day shows this peak is reduced with the fermentation time. It means that the macromolecules are almost completely decomposed into NAG, which proves that the main component of the Lentinula edodes stems fermentation product is NAG, and the reason is that the Lentinula edodes stems contain a large amount of chitin, and the Cordyceps militaris can secrete a chitin enzyme to decompose the chitin into NAG.

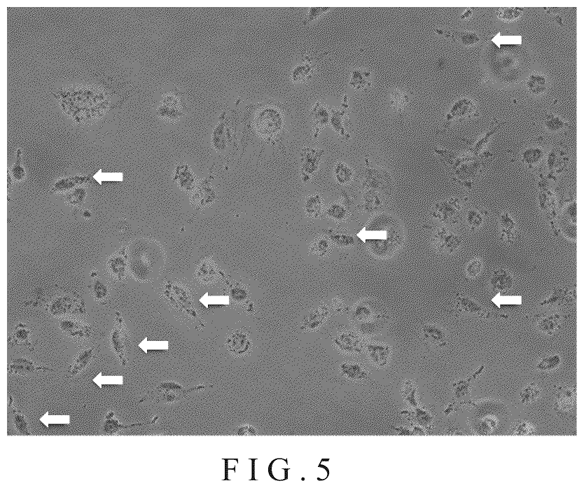

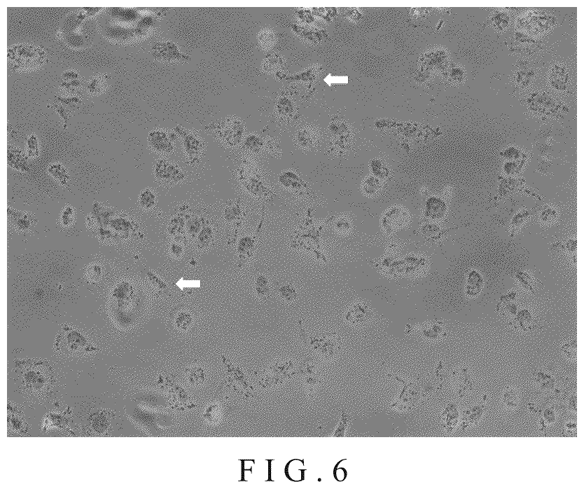

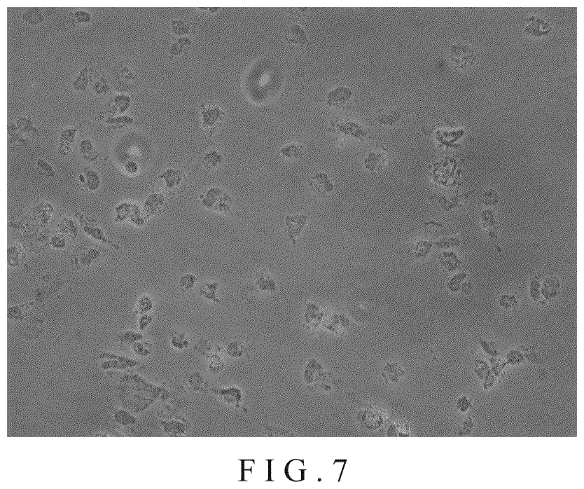

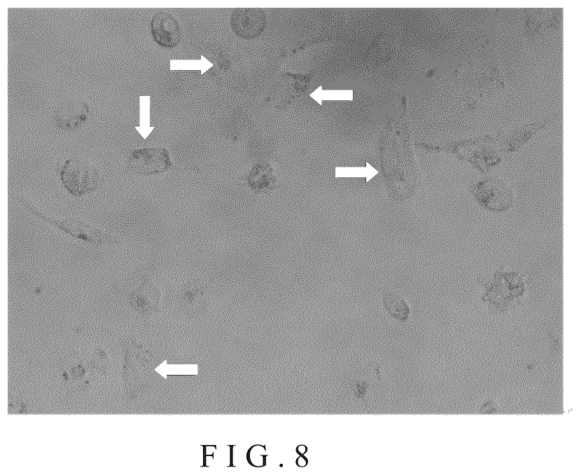

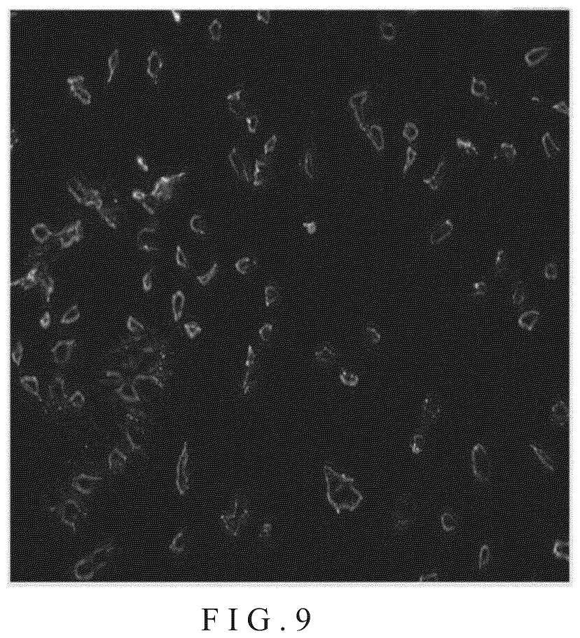

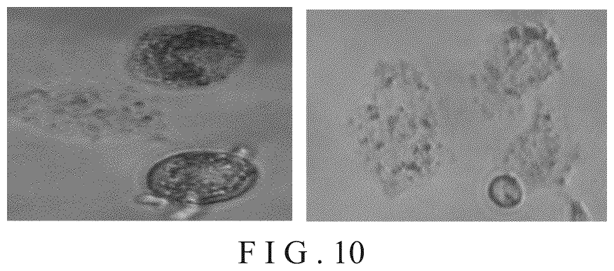

Referring to FIGS. 5-10, in order to prove that the Lentinula edodes stems fermentation product can be used to induce the mesenchymal epithelial transition, the Lentinula edodes stems fermentation product is used to culture the Huh7 human liver cancer cells, and the effect can be observed. First, the Huh7 human liver cancer cells are cultured in an EMEM culture medium containing 10% FCS (fetal calf serum) for 8 hours. Then, the Huh7 human liver cancer cells are cultured in another EMEM culture medium containing 0.1% FCS for 24 hours. The culture environment includes a cell culture box containing 5% CO2 under a temperature of 37° C. The cultured samples were divided into four groups. In one group, when the culture medium is changed, no other substance is added. In other three groups, when the culture medium is changed, the Lentinula edodes stems fermentation product with 5%, 10%, and 15% volume ratio is added. As shown in the figures, FIG. 5 is a photomicrograph of the Huh7 cells that are cultured without adding the Lentinula edodes stems fermentation product. As indicated by the arrows, the Huh7 cells have an elongated shape. FIGS. 6-8 respectively represent photomicrographs of the Huh7 cells that are cultured by 5%, 10% and 15% of the Lentinula edodes stems fermentation product. It is observed that the shape of the Huh7 cells is changed to become square and polygonal, as indicated by the arrows shown in FIG. 8. FIG. 9 is a fluorescent photomicrograph of the Huh7 cells that are cultured by 15% of the Lentinula edodes stems fermentation product wherein the E-cadherin is colored. It is observed that the expression of E-cadherin is obvious. FIG. 10 is a comparison photo of the Huh7 cells that are cultured for 8 hours, then added with 15% of the Lentinula edodes stems fermentation product, and then cultured for 24 hours. The right-side photograph shows the Lentinula edodes stems fermentation product is added and cultured for 24 hours. It is found that, the two ball-shaped cells on the right side become square, and the cells on the left are also changed from elongated shape to a square shape, which proves that the Lentinula edodes stems fermentation product actually induces the mesenchymal epithelial transition (MET), so that the Huh7 cells are changed into epithelial cells.

The above description is only a preferred embodiment of the present invention, and should not limit the scope of the present invention. All equivalent changes and modifications made according to the scope of the claims should still fall within the protection scope of the present invention.

1. A method for forming a fungi fermentation product containing N-acetylglucosamine to convert cancer cells into normal cells through a mesenchymal epithelial transition mechanism;
wherein:
the fungi fermentation product containing N-acetylglucosamine is added in a culture medium of cancer cells to induce and transform the cancer cells into epithelial cells;
the method for forming the fungi fermentation product comprises:
a fungus treatment procedure including preparing a fruiting body or mycelium of fungi crops with a predetermined weight and cleaning the fruiting body, then mixing the fungi crops with water, then crushing the fungi crops to form a fungi liquid, then heating and sterilizing the fungi liquid to form a fungi fruiting body or mycelium liquid;
a strain activation procedure including in turn culturing a Cordyceps fungus under a solid state and a liquid state successively to form a Cordyceps fungus liquid;
a fermentation procedure including mixing and culturing the fungi fruiting body or mycelium liquid and the Cordyceps fungus liquid under a temperature of 25° C. for 30 days to form the fungi fermentation product containing N-acetylglucosamine.

2. The method as claimed in claim 1, wherein the cancer cells are Huh7 cells.

3. The method as claimed in claim 1, wherein the fungi crops include Lentinula edodes, Agaricus bisporus, Agaricus campestris, Ganoderma lucidum, Flammulina velutipes or Auricularia auricula-judae.

4. The method as claimed in claim 1, wherein in the fungus treatment procedure, the fungi crops and the water are mixed with a weight ratio of 1:9.

5. The method as claimed in claim 1, wherein in the fungus treatment procedure, the fungi liquid is heated at a temperature of 121° C. during 1 hour.

6. The method as claimed in claim 1, wherein the Cordyceps fungus includes Cordyceps militaris or Ophiocordyceps sinensis.

7. The method as claimed in claim 1, wherein in the strain activation procedure includes placing the Cordyceps fungus in a PDA culture medium to perform a solid-state culture at 25° C. for 7 days, then placing the Cordyceps fungus in a liquid culture medium to perform a liquid-state culture at 25° C. for 10 days, under vibration with a rotation speed of 180 rpm, and the liquid culture medium has a composition including 20 g of glucose, 1 g of peptone, 20 g of agar and 20 g of malt extract per 1 L of distilled water.

8. The method as claimed in claim 1, wherein in the fermentation procedure, the fungi liquid and the Cordyceps fungus liquid are mixed with a weight ratio of 20:1.

9. The method as claimed in claim 1, wherein an addition ratio of the fungi fermentation product is 5-15% of a volume the culture medium.
